(19) United States
(12) Patent Application Publication
(10) Pub. No.: US 2005/0043296 A1
(43) Pub. Date: Feb. 24, 2005

(54) Compositions and methods for treating sexual dysfunction

(76) Inventors: Michael Hawley, Kalamazoo, MI (US); Mark J. Kontny, Libertyville, IL (US); Gordon W. Halstead, Scotts, MI (US)

Correspondence Address: HARNESS, DICKEY, & PIERCE, P.L.C, 7700 BONHOMME, STE 400, ST. LOUIS, MO, 63105, US

(21) Appl. No.: 10/678,212

(22) Filed: Oct. 2, 2003

Related U.S. Application Data
(60) Provisional application No. 60/416,294, filed on Oct. 4, 2002.

(51) Int. Cl. A61K 31/551; A61K 31/542; A61K 31/538; A61K 31/5377; A61K 31/496; A61K 31/4745

(52) U.S. Cl. 514/220; 514/224.5; 514/230.2; 514/250; 514/294; 514/234.2; 514/253.03

(57) ABSTRACT

A pharmaceutical composition comprising a salt or a crystalline salt in which the salt or crystalline salt comprises an acid of an artificial sweetener and a tricyclic compound is provided. Also provided are methods of making the salt, methods of making the crystalline salt, and methods of treatment of sexual dysfunction using the salt or the crystalline salt.

This application claims the benefit of U.S. Provisional Application No. 60/416,294, filed Oct. 4, 2002.

### BACKGROUND OF THE INVENTION

(1) Field Of The Invention

The present invention relates to tricyclic, nitrogen-containing compounds, which are heterocyclic amines, and salts thereof, and, in particular, to water-soluble salts of the compounds and an acid, which is an artificial sweetener. The compounds are suitable for use in rapidly dissolving, flavor masking formulations for treating sexual dysfunction or increasing sexual interest or performance.

(2) Description Of The Related Art

U.S. Pat. No. 5,273,975 and International Application Publication No. WO00/40226 disclose compounds useful in the treatment of sexual dysfunction. These compounds are tricyclic nitrogen-containing heterocyclic compounds of formula (I) as described hereinbelow.

U.S. Pat. No. 5,273,975 also describes salts of said compounds. The salts are, preferably, salts of an acid selected from hydrochloric acid, hydrobromic acid, hydroiodic acid, sulfuric acid, phosphoric acid, acetic acid, propionic acid, lactic acid, maleic acid, malic acid, succinic acid, tartaric acid, cyclohexanesulfamic acid, methanesulfonic acid, ethanesulfonic acid, benzenesulfonic acid, toluenesulfonic acid and other pharmaceutically acceptable acids that can provide a counter ion for an amine. The cyclohexanesulfamic acid is the acid form of the artificial sweetener cyclamate. However, crystals of a compound of formula (I) and cyclohexanesulfamic acid are not disclosed, nor does this refererence disclose the compound (R)-5,6-dihydro-5-(methylamino)-4H-imidazo[4,5-ij]-quinoline-2(1H)-thione or salts thereof. The useful dosage range provided in U.S. Pat. No. 5,273,975 is at least 10 mg up to about 1200 mg per day of the compound.

International Application Publication No. WO 00/40226 discloses compounds of formula (I) and salts thereof, that are effective in treating sexual disturbances at a dose of less than 8 mg and, in particular, about 0.2 to about 8 mg/person/dose, preferably, about 0.5 to about 5 mg/person/dose, more preferably, about 1 to about 3 mg/person/dose. This application, however, does not provide a rapid release formulation of a compound of formula (I). The salt disclosed in the '226 application is a salt of an acid and a compound of formula (I), in which the acid is preferably selected from methanesulfonic acid, hydrochloric acid, hydrobromic acid, sulfuric acid, phosphoric acid, nitric acid, benzoic acid, citric acid, tartaric acid, fumaric acid, maleic acid, CH₃—(CH₂)ₙ—COOH, where n is 0 thru 4, and HOOC—(CH₂)ₙ—COOH, where n is defined as above. None of the disclosed acids is an artificial sweetener.

International Publication WO 99/16442 and U.S. Pat. No. 6,197,339 B1 describe a maleate salt of (R)-5,6-dihydro-5-(methylamino)-4H-imidazo[4,5,1-ij]quinolin-2(1H)-one, a compound of formula (I). In addition, a crystal structure of the maleate salt of a compound of formula (I) is described in an article by Heier et al. (Heier, R. F., Dolak, L. A., Duncan, J. N., Hyslop, D. K., Lipton, M. F., Martin, I. J., Mauragis, M. A., Piercey, M. F., Nichols, N. F., Schreur, P. J., Smith, M. W., Moon, M. W. (1997) Synthesis and biological activities of (R)-5,6-dihydro-N,N-dimethyl-4H-imidazo[4,5,1-ij]quinolin-5-amine and its metabolites. J. Med. Chem. 40: 639-646). These references, however, do not disclose a salt of a compound of formula (I) and an artificial sweetener or a crystalline salt of a compound of formula (I) and an artificial sweetener, nor do they disclose a therapeutically effective dose of less than 8 mg/day. In addition, these references do not disclose the compound (R)-5,6-dihydro-5-(methylamino)-4H-imidazo[4,5-ij]-quinoline-2(1H)-thione or salts thereof.

International Publication WO 02/062315 describes a compound of formula (I), and in preferred embodiments, compounds of formula (II) 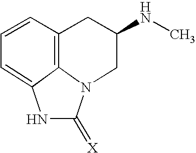
where X is O or S. Although some embodiments described in this reference include formulations comprising a mixture of a compound of formula (II) and a sweetener, the sweetener is preferably selected from sucrose, mannitol, propylene glycol, sodium saccharin, acesulfame K, neotame, and aspartame. A salt of a compound of formula (I) and an artificial sweetener is not disclosed, nor is a crystalline salt of a compound of formula (I) and an artificial sweetener disclosed, including a salt or crystalline salt of a compound of formula (I) and saccharinic acid or cyclamic acid.

### BRIEF DESCRIPTION OF THE INVENTION

In accordance with the present invention, the inventors herein have succeeded in discovering salts comprising artificial sweeteners and tricyclic compounds of formula (I). In preferred embodiments, the salts are substantially crystalline in form. The salts can be used in rapid release formulations that are particularly useful for treatment of sexual dysfunction.

Thus, in one embodiment, the invention comprises a pharmaceutical composition comprising a salt, the salt comprising an artificial sweetener and a compound of formula (I) 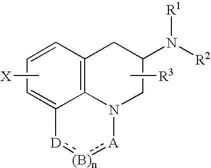
where
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NCHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where 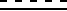
   is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
   A is CH, C—SCH₃, C—NH₂, C—NHCH₃, C—NHCOOH₃, C—NHCN, N; then D is CH, N;
(3) that when n is 1, and
   A is CH₂, CH-(halogen) where halogen is as defined above, CHCH₃, C═O, C—S , C═NH, SO₂; and
   B is CH₂, CH-(halogen) where halogen is as defined above, C═O, NH, N—CH₃; then
   D is CH₂, C═O, O, NH, N—CH₃;
(4) that when n is 1, and
   A is CH, C—SCH₃, C—NH₂, C—NHCH₃, C—NHCOOCH₃, C—NHCN, N; and
   B is CH, N; then
   D is CH₂, C═O, O, NH, N—CH₃;
(5) that when n is 1, and
   A is CH₂, CHCH₃, C═O, C═S, C═NH, SO₂, and
   B is CH, N; then
   D is CH, N.

In certain embodiments, the composition is a salt comprising an artificial sweetener and a compound of formula (I). In preferred embodiments, the compound is (R)-5,6-dihydro-5-(methylamino)-4H-imidazo[4,5-ij]-quinoline-2(1H)-thione. The artificial sweetener is any artificial sweetener that can be combined with the compound to form a salt, and is preferably selected from the group consisting of saccharinic acid and cyclamic acid.

In another embodiment, the composition comprises a crystalline salt, the crystalline salt comprising an acid of an artificial sweetener and a compound of formula (I). Preferably, the compound is (R)-5,6-dihydro-5-(methylamino)-4H-imidazo[4,5-ij]-quinoline-2(1H)-thione. The acid of the artificial sweetener of the crystalline salt is preferably cyclamic acid.

In another embodiment, the composition comprises a salt, the salt comprising an artificial sweetener and a compound of formula (I), wherein the composition has a sexually therapeutic effective amount as well as a sexually useful effective amount of from about 0.2 to about 8 mg/person/dose. In another embodiment, a composition of the present invention rapidly disintegrates when placed in an oral environment. The composition thereby provides a fast-melt medicament that is palatable or organoleptically acceptable to a recipient.

In one embodiment, the composition is in a dosage form comprising the crystalline salt and a pharmaceutically acceptable excipient. The dosage form is administered orally. The dosage form disintegrates rapidly upon intraoral administration, while having acceptable organoleptic properties. The dosage form preferably disintegrates in the mouth without the need for drinking water or other liquid, and provides a therapeutic, interest-enhancing or performance-enhancing effect within about 10 minutes to about 8 hours after administration.

Also provided by the present invention are methods of preparing the salts described herein. In addition, the present invention provides methods of preparing crystalline salt forms. The latter methods involve dissolving a compound of formula (I) and an acid of an artificial sweetener in an organic solvent to form a solution, and precipitating a crystalline salt form from the solution. The artificial sweetener is, preferably, cyclamic acid, and the organic solvent is, preferably, a mixture of tetrahydrofuran and methanol. In additional embodiments, dosage forms of the invention are provided that comprise a salt or a crystalline salt of the invention combined with one or more pharmaceutically acceptable excipients, as well as methods of preparing these dosage forms.

Also provided by the present invention are methods of use of compositions of the present invention for treatment of sexual dysfunction and for enhancement of sexual desire, interest or performance. In this embodiment, a therapeutically effective amount of a salt, preferably a crystalline salt, of a dosage form of the invention is placed in the oral cavity. Interactions of the dosage form with fluids in the oral cavity lead to rapid disintegration of the dosage form, leading to rapid absorption and consequent therapeutic or stimulatory effect.

Further areas of applicability of the present invention will become apparent from the detailed description provided hereinafter. It should be understood that the detailed description and examples, while indicating preferred embodiments of the invention, are intended for purposes of illustration only and are not intended to limit the scope of the invention.

### BRIEF DESCRIPTION OF THE DRAWINGS

FIG. 1 presents differential scanning calorimetry traces for the maleic acid (labeled as “maleate”) and cyclamic acid (labeled as “cyclamate”) crystalline salts of (R)-5,6-dihydro-5-(methylamino)-4H-imidazo[4,5-i,j]-quinoline-2(1H)-thione.

FIG. 2 presents powder x-ray diffraction patterns of the maleic acid (labeled as “maleate”) and cyclamic acid (labeled as “cyclamate”) crystalline salts of (R)-5,6-dihydro-5-(methylamino)-4H-imidazo[4,5-i,j]-quinoline-2(1H)-thione.

### DETAILED DESCRIPTION OF THE INVENTION

The present invention provides a composition which comprises a salt, the salt comprising a compound of formula (I) and a water soluble acid which is an artificial sweetener. A salt of the invention is of high water solubility and has an acceptable taste, thus providing a rapid onset pharmaceutical composition useful for treatment of sexual dysfunction, stimulation of sexual activity and enhancement of sexual desire, interest, and performance in men and women. The compositions dissolve rapidly when placed in the environment of the oral cavity, thereby providing a fast-melt medicament which is rapidly absorbed.

A composition of the present invention is effective in treating sexual dysfunction at a dose of less than about 8 mg and, in particular, about 0.2 to about 8 mg/person/dose, preferably, about 0.5 to about 5 mg/person/dose, more preferably, about 1 to about 3 mg/person/dose. As used herein, dosage amounts refer to the amount of the pharmaceutical compound rather than the amount of a salt or a composition comprising the compound. For example, a dosage of “8 mg” means a dosage of 8 mg of the compound, rather than 8 mg of a salt comprising an acid and the compound.

The artificial sweetener acid which provides the anion of the salt comprising formula (I) masks the taste of the compound of formula (I), thereby obviating the need to administer compounds of formula (I) in a tablet or capsule to avoid the unpleasant taste of the compound. A further benefit of the invention is that the low dosage required for a pharmaceutical effect reduces undesired side effects of the medication. Yet another benefit of the invention is that rapid dissolution of a dosage form provides rapid onset of action following administration, for example rapid dissolution of a dosage form following placement in the oral cavity.

Rapid dissolution of a composition is defined herein as dissolution of a therapeutically effective amount or a sexual-stimulatory effective amount of a pharmaceutical compound within about four minutes of contact between a dosage form of the composition and fluid within the oral cavity.

Administration of the present invention preferably comprises administration of the compositions to a human subject, although administration can also be to a non-human mammal. Non-human mammals include commercial animals, (including non-limiting examples such as horses, cattle, swine, sheep and transgenic mice) and exotic animal such as zoo animals, sporting animals (including non-limiting examples such as horses and dogs) as well as companion animals (including non-limiting examples such as dogs and cats).

As used herein, a “therapeutically effective amount” is an amount sufficient to improve sexual desire, interest, or performance in a subject experiencing sexual dysfunction. Such desire, interest, or performance relates to sexual intercourse whether or not accompanied by orgasm, ejaculation, masturbation, and or sexual foreplay. A “sexual-stimulatory effective amount” is an amount sufficient to improve sexual desire, interest or performance in a subject whether or not the subject has a sexual dysfunction condition. A dosage form having “acceptable organoleptic properties” herein is one that, upon administration, provide sensations that are not as perceived offensive or objectionable by a majority of subjects. In preferred embodiments, administration is intraoral in an amount providing a single dose of a therapeutic agent, which provides a taste, smell, and/or mouth feel which is not perceived offensive or objectionable by a majority of subjects.

In a preferred embodiment, the invention comprises a salt comprising an artificial sweetener and a compound of formula (I). Preferably, the compound is (R)-5,6-dihydro-5-(methylamino)-4H-imidazo[4,5-ij]-quinoline-2(1H)-thione. The artificial sweetener is preferably selected from the group consisting of cyclamate, saccharinate, aspartame, neotame, acesulfame, alitame and combinations thereof. More preferably, the artificial sweetener is saccharinate or cyclamate. It is understood that a salt of the present invention combines a negatively charged form of an artificial sweetener acid and a positively charged form of the pharmaceutical compound in approximately equimolar amounts. However, in some embodiments, the molar ratio of the artificial sweetener to that of the compound is more or less than 1:1, so that a free base form of the compound coexists with the salt, to adjust the taste of the dosage form.

In another preferred embodiment, the invention comprises a crystalline salt, the salt comprising an artificial sweetener and a compound of formula (I). Preferably, the crystalline salt is the cyclamic acid salt of (R)-5,6-dihydro-5-(methylamino)-4H-imidazo[4,5-ij]-quinoline-2(1H)-thione.

In preferred embodiments, the amount of the salt is lower than an amount causing significant side-effects. In the present invention, dosage amounts of the compounds of formula (I) lower than 10 mg do not lead to significant side-effects. Preferably, a dosage form of the salt of a compound of formula (I) is lower than 10 mg, more preferably about 8 mg or less.

The compounds of the present invention are preferably in a pharmaceutical composition containing unit doses. By unit dose or unit dosage form, it is meant that the active compound is present in a discrete, known amount in the pharmaceutical composition. Such unit doses are effective in treating conditions of sexual disturbance or in improving sexual performance. The pharmaceutical compositions of the present invention, in unit dosage form, comprise an amount of from about 0.05 mg to about 8 mg of the compound of formula (I), preferably about 0.1 to about 3 mg of the compound of formula (I), and more preferably about 0.25 mg to about 1 mg of the compound of formula (I).

The precise dosage of a compound of formula (I) which is useful in the treatment of sexual disturbances or to improve or increase sexual interest or performance will depend upon the route of administration, the particular compound used, the particular condition being treated, the severity of the condition being treated, the age, weight, general physical condition of the particular patient and the like as can be readily determined by the skilled artisan.

The present invention includes, in some embodiments, dosage forms for peroral administration, for example tablets and capsules. Any methods that are known in the art can be used to prepare these dosage forms. Such dosage forms can comprise, in addition to a salt or a crystalline salt of the present invention, one or more components that are known in the art. Non-limiting examples of additional components are natural polymers (such as a protein or a starch), modified natural polymers (for example gelatin), edible oils, and sugars.

The compositions of the present invention are preferably administered in dosage forms suitable for intraoral administration, for example:
buccal and sublingual tablets including those permitting absorption of the therapeutic agent through the oral mucosa;
chewable tablets;
rapidly disintegrating oral dosage forms such as fast-melt tablets;
lozenges and pastilles including those permitting oropharyngeal absorption of the therapeutic agent;
breath-fresheners such as breath-mints;
chewing gums and chewing candy;
lollipops and popsicles;
food adjuncts, such as broths, bouillon cubes and granules, puddings, jellies, and spreads;
candies and chocolates;
periodontal gels;
mouthwashes;
oral and nasal drops and sprays;
dosage forms adapted for inhalation as an aerosol or vapor;
elixirs, solutions, suspensions and other orally administered liquid dosage forms;
powders, granules and tablets for dissolution or dispersion in water prior to oral administration; and
effervescent tablets and granules.

A fast-melt formulation of the invention contains, in some embodiments one or more additional pharmaceutically acceptable excipients, for example wetting agents, water-soluble lubricants, water-insoluble lubricants, disintegrants, glidants, sweeteners, flavoring agents, effervescent agents and colorants. Such additional components are physically and chemically compatible with the other ingredients of the dosage form. Suitable excipients for fast-melt pharmaceutical formulations are well known in the art. Preferred embodiments of the invention include a moldable mixture comprising crystals of the cyclamate salt of (R)-5,6-dihydro-5-(methylamino)-4H-imidazo[4,5-i,j]-quinoline-2(1H)-thione, and at least one excipient. Examples of fast-melt formulations and processes for preparing such formulations, are independently disclosed in patents and publications individually cited immediately below.
U.S. Pat. No. 3,885,026 to Heinemann et al.
U.S. Pat. No. 4,134,943 to Knitsch et al.
U.S. Pat. No. 4,305,502 to Gregory et al.
U.S. Pat. No. 4,371,516 to Gregory et al.
U.S. Pat. No. 4,414,198 to Michaelson.
U.S. Pat. No. 4,855,326 to Fuisz.
U.S. Pat. No. 4,946,684 to Blank et al.
U.S. Pat. No. 5,073,374 to McCarty.
U.S. Pat. No. 5,178,878 to Wheling et al.
U.S. Pat. No. 5,298,261 to Pebley et al.
U.S. Pat. No. 5,401,514 to Juch et al.
U.S. Pat. No. 5,417,985 to Coutel et al.
U.S. Pat. No. 5,464,632 to Cousin et al.
U.S. Pat. No. 5,466,464 to Masaki, et al.
U.S. Pat. No. 5,082,667 to Van Scoik.
U.S. Pat. No. 5,501,861 to Makino et al.
U.S. Pat. No. 5,503,846 to Wehling et al.
U.S. Pat. No. 5,518,730 to Fuisz.
U.S. Pat. No. 5,576,014 to Mizumoto et al.
U.S. Pat. No. 5,587,172 to Cherukuri et al.
U.S. Pat. No. 5,587,180 to Allen et al.
U.S. Pat. No. 5,607,697 to Alkire et al.
U.S. Pat. No. 5,622,719 to Myers et al.
U.S. Pat. No. 5,653,926 to Bogue & Myers.
U.S. Pat. No. 5,662,849 to Bogue et al.
U.S. Pat. No. 5,733,577 to Myers et al.
U.S. Pat. No. 5,762,961 to Roser et al.
U.S. Pat. No. 5,807,576 to Allen et al.
U.S. Pat. No. 5,837,285 to Nakamichi et al.
U.S. Pat. No. 5,869,098 to Misra et al.
U.S. Pat. No. 5,876,759 to Gowan.
U.S. Pat. No. 5,939,091 to Eoga et al.
U.S. Pat. No. 5,958,453 to Ohno et al.
U.S. Pat. No. 6,010,719 to Remon et al.
U.S. Pat. No. 6,024,981 to Khankari et al.

Approaches to formulating fast-melt tablets, including several in the above-cited patents, have been summarized by Chang et al. in Pharmaceutical Technology, June 2000, pp. 52-58.

An excipient of the present invention preferably comprises one or more pharmaceutically acceptable carbohydrates. The carbohydrate is selected from natural and modified celluloses, for example microcrystalline cellulose, methylcellulose, ethylcellulose, hydroxypropylcellulose, hydroxypropylmethylcellulose, carboxymethycellulose, and sodium carboxymethylcellulose; natural and modified starches, for example corn starch, pregelatinized starch, and sodium starch glycolate; and mono-, di- and oligosaccharides comprising up to 6 saccharide units, including sugars and sugar alcohols, for example erythritol, glucose, lactose, maltitol, maltose, mannitol, sorbitol, sucrose and xylitol. It is preferred that at least one carbohydrate substantially present in the carrier system is selected from sugars and sugar alcohols, more preferably those exhibiting rapid dissolution in the mouth, most preferably those exhibiting rapid dissolution and providing a sweet taste. Preferably, saccharides of the invention are mono- or disaccharides, and are preferably selected from sugars and sugar alcohols having high moldability, e.g., maltitol, maltose and sorbitol, as well as sugars and sugar alcohols having low moldability, particularly when in finely particulate as opposed to granular form, for example, glucose, lactose, mannitol, sucrose and xylitol.

Carbohydrates of preferred embodiments of the invention are present in a fast-melt formulation of the invention in a total amount of about 20% to about 95% by weight of the formulation. Preferably, carbohydrates comprise at least about 50% (w/w) of the formulation.

Pharmaceutically acceptable wetting agents can be included in certain embodiments of a fast-melt formulation of the invention. Non-limiting examples of wetting agents include, individually or in combination, surfactants, hydrophilic polymers and certain clays. Non-limiting examples of pharmaceutically acceptable surfactants include: quaternary ammonium compounds, for example benzalkonium chloride, benzethonium chloride and cetylpyridinium chloride, and dioctyl sodium sulfoccinate; polyoxyethylene alkylphenyl ethers, for example, nonoxynol 9, nonoxynol 10, and octonynol 9, poloxamers (polyoxyethylene and polyoxypropylene block copolymers); polyoxyethylene fatty acid glycerides; oils, for example polyoxyethylene (8) caprylic/capric mono- and diglycerides, polyoxyethylene (35) castor oil and polyoxyethylene (40) hydrogenated castor oil; polyoxyethylene alkyl ethers, for example polyoxyethylene (20) cetostearyl ether; polyoxyethylene fatty acid esters, for example, polyoxyethylene (40) stearate; polyoxyethylene sorbitan esters, for example, polysorbate 20 and polysorbate 80; propylene glycol fatty acid esters, for example, propylene glycol laurate, sodium lauryl sulfate; fatty acids and salts thereof, for example, oleic acid, sodium oleate and triethanolamine oleate; glyceryl fatty acid esters, for example glyceryl monostearate; sorbitan esters, for example, sorbitan monolaurate, sorbitan monooleate, sorbitan monopalmitate, sorbitan monostearate, tyloxapol, and mixtures thereof. Preferably, the wetting agent is sodium lauryl sulfate. One or more wetting agents can be present in a formulation of the invention in a total amount of about 0.05% to about 5% (w/w) of the formulation.

In certain embodiments of the invention, pharmaceutically acceptable water-soluble lubricants are also present. Non-limiting examples of lubricants include boric acid, sodium benzoate, sodium acetate, sodium fuimarate, sodium chloride, DL-leucine, polyethylene glycol (for example, Carbowax™ 4000 and Carbowax™ 6000), sodium oleate and mixtures thereof. In preferred embodiments, one or more water-soluble lubricant is present in a total amount of about 0.05% to about 5% (w/w) of the formulation.

In certain embodiments of the invention, one or more pharmaceutically acceptable disintegrants are also present. Non-limiting examples of a disintegrant include: starch; sodium starch glycolate; clay, such as Veegum™ HV; cellulose, such as purified cellulose, methylcellulose, sodium carboxymethylcellulose, carboxymethylcellulose; croscarmellose sodium; alginates; pregelatinized corn starches, such as National™ 1551 and National™ 1550; crospovidone; gums, such as agar, guar, locust bean, karaya, pectin, and tragacanth gums; and mixtures thereof. Preferably, the disintegrant is selected from croscarmellose sodium, sodium starch glycolate and mixtures thereof. One or more disintegrants can be present in a formulation of the invention in a total amount of about 0.5% to about 7.5% (w/w) of the formulation.

Certain embodiments of the invention include one or more pharmaceutically acceptable effervescent salt as a disintegrant and/or to enhance the organoleptic properties of a fast-melt formulation of the invention.

Certain embodiments of the invention include one or more pharmaceutically acceptable glidant. Non-limiting examples of glidants include silicon dioxide products such as fumed silica (for example, Cab-O-Sil™ of Cabot Corp. and Aerosil™ of Degussa).

In certain embodiments of the invention, pharmaceutically acceptable natural or artificial sweeteners are also included (in addition to the artificial sweetener of the salt). Non-limiting examples of such sweeteners are sucrose, mannitol, propylene glycol, sodium saccharinate, acesulfame K, neotame, and aspartame. Flavoring agents are also used in some embodiments. Non-limiting examples of pharmaceutically acceptable flavoring agents include peppermint, spearmint, grape, cherry, strawberry, and lemon.

In a preferred embodiment, the formulation is an oral fast-melt tablet or an oral fast-melt sheet. Tablets can be of any suitable dimensions. Tablets are preferably 8 mm to 12 mm in diameter or long axis, and can be of any shape, for example, round, elliptical, oblong, spheroidal, or polygonal. Tablets of the invention can have etchings or monograms on one or both sides. In addition, tablets can be scored or otherwise provided with means for convenient breaking into unit-dose segments, but a tablet is preferably a self-contained dosage form delivering a single unit dose.

In one embodiment of the present invention, the formulation is a rapidly water-disintegratable tablet comprising a crystalline salt, the crystalline salt comprising an artificial sweetener, preferably cyclamate, and a compound of formula (I), preferably (R)-5,6-dihydro-5-(methylamino)-4H-imidazo[4,5-ij]-quinoline-2(1H)-thione, and having distributed therewithin a disintegrating system comprising an unreacted, intimate mixture of alginic acid and a water soluble metal carbonate in proportions reactive to form an alginic acid salt and carbonic acid when the tablet is placed in water, substantially as disclosed in U.S. Pat. No. 4,414,198.

In another embodiment of the invention, the formulation comprises a cotton candy-like mass of spun fibers of a readily water-soluble material, for example, a sugar such as sucrose, fructose, dextrose, mannitol, sorbitol, lactose, or maltose, and/or a cellulosic material, for example methylcellulose, ethylcellulose, hydroxymethylcellulose, hydroxyethylcellulose, alkali metal salts of carboxymethylcellulose, and a salt comprising an artificial sweetener and a compound of formula (I), preferably (R)-5,6-dihydro-5-(methylamino)-4H-imidazo[4,5-ij]-quinoline-2(1H)-thione. Preferably, the artificial sweetener is selected from the group consisting of cyclamate and saccharin. Preferably, the salt is distributed on or incorporated in the mass of spun fibers, substantially as disclosed in U.S. Pat. No. 4,855,326.

The invention includes methods for making a dosage form of a salt comprising a compound of formula (I) and an artificial sweetener. In accordance with the methods, a compound of formula (I) and an acid of an artificial sweetener are both dissolved in a suitable organic solvent. This is followed by removal of the solvent. The solvent can be any solvent capable of solubilizing both the compound and the sweetener, preferably a pharmaceutically acceptable solvent. The removing preferably comprises evaporating the solvent, and/or allowing spontaneous precipitation from solution. In the latter case, solvent is separated from the precipitate by standard procedures well known to those of skill in the organic chemistry art, preferably filtration.

The precipitated material can be in crystalline form. In certain embodiments, crystalline material is formed when a salt comprising cyclarnic acid and a compound of formula (I) is prepared. In accordance with this method, a solvent mixture comprising organic solvents tetrahydrofuran and methanol is prepared and added to the compound of formula (I) thereby dissolving the compound. Cyclamic acid is also added to the solvent mixture, thereby forming a solution comprising cyclamic acid and the compound of formula (I). The molar ratio of cyclamic acid to the compound of formula (I) in the solvent mixture is about 1:5 to about 5:1, preferably about 1:2 to about 2:1, more preferably about 1:1.4 to about 1.4:1. Crystals comprising a salt comprising cyclamic acid and a compound of formula (I) are then formed from the solution upon stirring at ambient temperature. These crystals can be separated from the organic solvents by methods well known in the art, for example by filtration. A compound of formula (I) which comprises the crystalline cyclamate salt is, illustratively, (R)-5,6-dihydro-5-(methylamino)-4H-imidazo[4,5-ij]-quinoline-2(1H)-thione.

In contrast, a glassy solid is formed when the saccharin salt of a compound of formula (I) is prepared. In accordance with this method, a solvent mixture of tetrahydrofuran and methanol is prepared and added to the compound of formula (D) thereby dissolving the compound. Saccharin is also added to the solvent mixture in an amount approximately equimolar to that of the compound. Solvent can be removed by rotovapping, thereby yielding a glassy solid. A compound of formula (I) which forms a glassy solid, non-crystalline salt of saccharin is, illustratively (R)-5,6-dihydro-5-(methylamino)-4H-imidazo[4,5 -ij]-quinoline-2( 1H)-thione.

In embodiments including a moldable mixture comprising a salt or a crystalline salt of the present invention and at least one excipient, the mixture is preferably formed into a solid dosage form such as a tablet, lozenge or wafer using techniques well known to those of skill in the art. In a particular embodiment, tablets comprising a salt of the invention are prepared by kneading a mixture of the salt and a readily water-soluble crystalline or powdery solid, preferably one having a sweet taste, for example sucrose, lactose, glucose, fructose, xylitol, sorbitol, or mannitol, with a suitable amount of water, typically about 1% to about 10% by weight of the solid components. The wet kneaded mixture is then shaped, compressed, and dried to form a solid tablet substantially as disclosed in U.S. Pat. No. 5,837,285.

In certain embodiments, the salts and crystalline salts of the present invention are used by administering a dosage form of the salt to a person in need thereof in anticipation of or during sexual activity. In preferred embodiments, the individual receiving the dosage form in his or her mouth dissolves the salt by contacting the salt with a liquid in the mouth, such as saliva or an externally supplied liquid such as water, a soft drink, a juice, or an alcoholic beverage. Dissolved compound of the invention is then absorbed by standard physiological routes, for example by swallowing or absorption from the oral cavity. Disintegration can be accelerated by mechanical manipulation in the mouth, for example by chewing or sucking at a tablet. Preferably, the dosage form is positioned sublingually or buccally, and is preferably dissolved by contact with saliva.

The timing and dosage of administration is determined by the user and/or a medical caregiver. Preferably, a dosage form of the invention is administered from about 10 minutes to about 8 hr prior to sexual activity. It is preferred that a dosage form of the invention be administered from about 30 to about 60 minutes prior to sexual activity. It is more preferred that a dosage form of the invention be administered about 30 minutes prior to sexual activity. Sexual activity includes sexual intercourse, with or without orgasm, ejaculation, masturbation, or sexual foreplay.

Methods of making salts of the invention, and analytical investigations of physical properties of the salts are illustrated in the examples provided below.

### EXAMPLE 1

This example illustrates the formation of a crystalline cyclamate salt of (R)-5,6-dihydro-5-(methylamino)-4H-imidazo[4,5-ij]-quinoline-2(1H)-thione.

Sixty-four (64) mg of (R)-5,6-dihydro-5-(methylamino)-4H-imidazo[4,5-ij]-quinoline-2(1H)-thione were placed in a 4 ml vial. One ml of tetrahydrofuran (THF) and one ml of methanol were added, producing a cloudy solution. The solution was filtered, yielding a yellow-brown clear solution. Thirty-six (36) mg of cyclamic acid was then added to the solution (i.e., molar ratio of about 1:0.7 (R)-5,6-dihydro-5-(methylamino)-4H-imidazo[4,5-ij]-quinoline-2(1H)-thione : cyclamic acid). Crystals then formed upon stirring at ambient temperature. The crystals of (R)-5,6-dihydro-5-(methylamino)-4H-imidazo[4,5-ij]-quinoline-2(1H)-thione cyclamate were collected by filtration, 57 mg total (about 76% yield).

### EXAMPLE 2

This example illustrates the preparation of a glassy solid saccharine salt of (R)-5,6-dihydro-5-(methylamino)-4H-imidazo[4,5-ij]-quinoline-2(1H)-thione saccharinate.

Sixty-one (61) mg of (R)-5,6-dihydro-5-(methylamino)-4H-imidazo[4,5-ij]-quinoline-2(1H)-thione was dissolved in 2 ml of methanol and 1 ml THF in a 20 ml roundbottom flask. Fifty (50) mg of saccharin was then added to the solution. Solvent was removed by evaporation with the aid of a rotary evaporator, yielding a glassy solid. Further attempts at crystallizing the material from acetonitrile, acetone, or THF did not lead to formation of crystals.

### EXAMPLE 3

This example illustrate physical properties of the crystalline cyclamate and maleate salts of (R)-5,6-dihydro-5-(methylamino)-4H-imidazo[4,5-ij]-quinoline-2(1H)-thione.

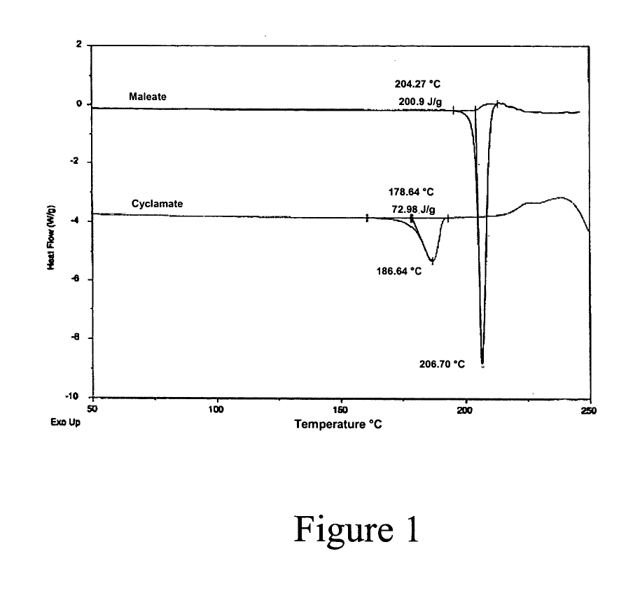

FIG. 1 presents differential scanning calorimetry traces of the crystalline cyclamate salt prepared in Example 1 and a comparably prepared maleate salt. The traces indicate that both materials have relatively high melting points, about 175° C. for the cyclamate salt and about 210° C. for the maleate salt. In addition, the sharpness of the curves suggests that both materials are substantially pure and substantially crystalline.

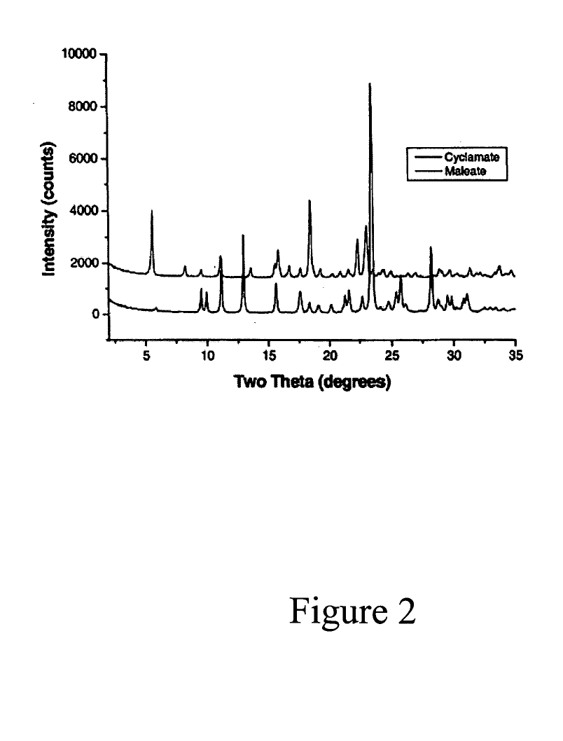

FIG. 2 presents powder x-ray diffraction patterns of the cyclamate and maleate salts. The x-ray diffraction profiles were obtained by standard techniques well known to those of skill in the art. The sharpness and heights of the peaks indicate that both materials are crystalline.

Table 1 presents analytical results for crystals of the cyclamate salt of (R)-5,6-dihydro-5-(methylamino)-4H-imidazo[4,5-ij]-quinoline-2(1H)-thione described in Example 1. Because the material obtained from the saccharinate salt was non-crystalline, no comparable studies were conducted.

TABLE 1

| Assay | Calculated | Result |
|---|---|---|
| Carbon | 51.23 | 51.17 |
| Hydrogen | 6.58 | 6.66 |
| Nitrogen | 14.06 | 13.93 |
| IR Spectra | | Supports structure |
| NMR | | Supports structure |
| HPLC | | 99.72% |
| ROI | | 0.60% |

As various changes could be made in the above methods and compositions without departing from the scope of the invention, it is intended that all matter contained in the above description be interpreted as illustrative and not in a limiting sense.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinency of the cited references.

1. A pharmaceutical composition in unit dosage form comprising a salt, the salt comprising an acid of an artificial sweetener and a compound of formula (I): 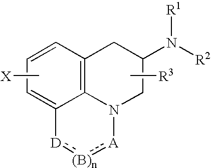
wherein
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where 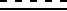
is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S , C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N,
the unit dosage form comprising from about 0.05 mg to no more than about 8 mg of the compound of formula (I).

2. A pharmaceutical composition according to claim 1, wherein the artificial sweetener is selected from the group consisting of cyclamate, saccharin, aspartame, neotame, acesulfame, alitame and combinations thereof.

3. A pharmaceutical composition according to claim 2, wherein the artificial sweetener is cyclamate.

4. A pharmaceutical composition according to claim 2, wherein the artificial sweetener is saccharin.

5. A pharmaceutical composition according to claim 1, wherein the compound of formula (I) is (R)-5,6-dihydro-5-(methylamino)-4H-imidazo[4,5-ij]-quinoline-2(1H)-thione.

6. A pharmaceutical composition according to claim 1, wherein the unit dosage form comprises from about 0.1 mg to about 3 mg of the compound of formula (I).

7. A pharmaceutical composition according to claim 6, wherein the unit dosage form comprises from about 0.25 mg to about 1 mg of the compound.

8. A pharmaceutical composition according to claim 1 comprising a crystalline salt, the crystalline salt comprising cyclamic acid and a compound of formula (I).

9. A pharmaceutical composition according to claim 8, wherein the compound of formula (I) is (R)-5,6-dihydro-5-(methylamino)-4H-imidazo[4,5-i,j]-quinoline-2(1H)-thione.

10. A pharmaceutical composition according to claim 9, which is in a unit dosage form comprising a dosage form comprising from about 0.05 mg to about 8 mg of the compound.

11. A pharmaceutical composition according to claim 10, which is in a unit dosage form comprising from about 0.1 mg to about 3 mg of the compound.

12. A pharmaceutical composition according to claim 11, which is in a unit dosage form comprising from about 0.25 mg to about 1 mg of the compound.

13. A method for treating sexual dysfunction in a subject, the method comprising orally administering to the subject a pharmaceutical composition in a unit dosage form comprising a salt, the salt comprising an acid of an artificial sweetener and a compound of formula (I): 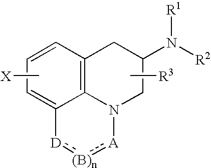
wherein
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, hd 1-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where 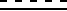
is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S , C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N,
the unit dosage form comprising from about 0.05 mg to no more than about 8 mg of the compound of formula (I).

14. A method according to claim 13 wherein the artificial sweetener is selected from the group consisting of cyclamate, saccharin, aspartame, neotame, acesulfame, alitame and combinations thereof.

15. A method according to claim 14, wherein the artificial sweetener is cyclamate.

16. A method according to claim 14, wherein the artificial sweetener is saccharin.

17. A method according to claim 13, wherein the compound of formula (I) is (R)-5,6-dihydro-5-(methylamino)-4H-imidazo[4,5-ij]-quinoline-2(1H)-thione.

18. A method according to claim 13, wherein the unit dosage form comprising from about 0.1 mg to about 3 mg of the compound of formula (I).

19. A method according to claim 18, wherein the unit dosage form comprises from about 0.25 mg to about 1 mg of the compound.

20. A method according to claim 13, wherein the pharmaceutical composition comprises a crystalline salt, the salt comprising cyclamic acid and a compound of formula (I).

21. A method according to claim 20, wherein the compound is (R)-5,6-dihydro-5-(methylamino)-4H-imidazo[4,5-ij]-quinoline-2(1H)-thione.

22. A method according to claim 21, wherein the pharmaceutical composition in unit dosage form comprises a dosage form comprising from about 0.05 mg to about 8 mg of the compound.

23. A method according to claim 22, wherein the unit dosage form comprising from about 0.05 mg to no more than about 8 mg of the compound of formula (I) is a unit dosage form comprising from about 0.1 mg to about 3 mg of the compound of formula (I).

24. A method according to claim 23, wherein the pharmaceutical composition in a unit dosage form comprises a dosage form comprising from about 0.25 mg to about 1 mg of the compound.

25. A method of making a crystalline salt, the crystalline salt comprising cyclamic acid and a compound of formula (I), the method comprising forming a solution comprising the compound of formula (I), the cyclamic acid, tetrahydrofuran and methanol, and forming the crystalline salt from the solution, wherein the compound of formula (I) is 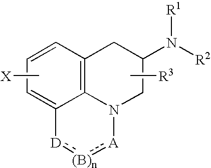
where
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where 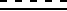
is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S , C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N.

26. A method according to claim 25, wherein the compound is (R)-5,6-dihydro-5-(methylamino)-4H-imidazo[4,5-i,j]-quinoline-2(1H)-thione.

27. A method according to claim 25, wherein the dissolved compound of formula (I) and the cyclamic acid added to the solution are in a molar ratio of about 1:2 to about 2:1.

28. A method according to claim 27, wherein, the dissolved compound of formula (I) and the cyclamic acid added to the solution are in a molar ratio of about 1:1.4 to about 1.4:1.

29. A method of increasing sexual desire, interest or performance in a human who is desirous thereof, the method comprising orally administering to the human a pharmaceutical composition in unit dosage form comprising a salt, the salt comprising an acid of an artificial sweetener and a compound of formula (I): 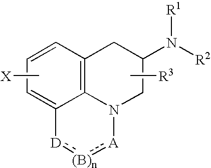
wherein
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where 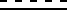
is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S , C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N,
the unit dosage form comprising from about 0.05 mg to no more than about 8 mg of the compound of formula (I).

30. A method according to claim 29 wherein the artificial sweetener is selected from the group consisting of cyclamate, saccharin, aspartame, neotame, acesulfame, alitame and combinations thereof.

31. A method according to claim 30, wherein the artificial sweetener is cyclamate.

32. A method according to claim 30, wherein the artificial sweetener is saccharin.

33. A method according to claim 29, wherein the compound of formula (I) is (R)-5,6-dihydro-5-(methylamino)-4H-imidazo[4,5-ij]-quinoline-2(1H)-thione.

34. A method according to claim 29, wherein the unit dosage form comprises from about 0.1 mg to about 3 mg of the compound of formula (I).

35. A method according to claim 34, wherein the unit dosage form comprises from about 0.25 mg to about 1 mg of the compound of formula (I).

36. A method according to claim 29, wherein the pharmaceutical composition comprises a crystalline salt, the crystalline salt comprising cyclamic acid and a compound of formula (I).

37. A method according to claim 36, wherein the compound is (R)-5,6-dihydro-5-(methylamino)-4H-imidazo[4,5-i,j]-quinoline-2(1H)-thione.

38. A method according to claim 37, wherein the pharmaceutical composition is in a unit dosage form, the dosage form comprising from about 0.05 mg to about 8 mg of the compound.

39. A method according to claim 38, wherein the unit dosage form comprises from about 0.1 mg to about 3 mg of the compound.

40. A method according to claim 39, wherein the pharmaceutical composition is in a unit dosage form comprising from about 0.25 mg to about 1 mg of the compound.
